(19) United States
(12) Patent Application Publication
(10) Pub. No.: US 2019/0175776 A1
(43) Pub. Date: Jun. 13, 2019

(54) INDICATOR AND DETECTION COMPOSITION FOR DISINFECTION COMPLIANCE MONITORING, INDICATOR AND DETECTION SYSTEM AND METHOD OF USING

(71) Applicant: Microban Products Company, Huntersville, NC (US)

(72) Inventors: Mai Le Phuong Ha, Cornelius, NC (US); Karen Terry Welch, Kannapolis, NC (US); Bruno Michael Mourao, Cornelius, NC (US); Gina Parise Sloan, Statesville, NC (US); Kevin Andrew Kavchok, Iron Station, NC (US)

(21) Appl. No.: 16/211,872

(22) Filed: Dec. 6, 2018

Related U.S. Application Data
(60) Provisional application No. 62/597,074, filed on Dec. 11, 2017.

(51) Int. Cl. A61L 2/28

(52) U.S. Cl. CPC A61L 2/28

(57) ABSTRACT

An indicator and detection composition having at least one xanthene dye, and at least one component being a pH indicator. A system and methods of using are provided.

### CROSS-REFERENCE TO RELATED APPLICATIONS

This application claims priority from U.S. Provisional Patent Application No. 62/597,074, filed on Dec. 11, 2017, in the United States Patent and Trademark Office. The disclosure of which is incorporated herein by reference in its entirety.

### FIELD OF THE INVENTION

The present invention relates to an indicator and detection composition and system for disinfection compliance monitoring, more particularly to a wet-state indicator and detection composition and system for disinfection compliance monitoring.

### BACKGROUND OF THE INVENTION

Multiple chemistries are utilized for disinfection and sterilization of surfaces. The two most common chemistries are quaternary ammonium and sodium hypochlorite. These are employed in consumer, professional, food service and healthcare applications. There are limited means to determine if the appropriate chemistry was utilized, if the chemistry was applied appropriately, or if there is residual chemistry providing extended protection.

Present technologies work to detect only one compound, quaternary ammonium or cationic species. Sodium hypochlorite is a typical disinfectant that is not captured in either of the above classifications. A third class of disinfectants typically employed is hydrogen peroxide (H₂O). Furthermore, hospitals typically quarantine patients with Clostridium difficile infections and need to monitor the use of the appropriate disinfectant, sodium hypochlorite. There is currently no way to monitor the use of disinfectants in a single package. The ability to quickly and uniquely identify the disinfectant chemistry utilized on a surface allows multiple industries to track compliance with cleaning protocols.

Current products on the market utilize a dry state indicator to detect that enough quaternary ammonium is present in a prepared solution (wet state). These products are not able to differentiate between chemistries.

For example, in some kits, an indicator and a developer must be packaged separately for stability. Furthermore, such kits are only able to detect the presence of quaternary ammonium.

Thus, there is a need for a device that overcomes the disadvantages associated with other detection kits.

### SUMMARY OF THE INVENTION

The present invention relates to an indicator and detection composition for disinfection compliance monitoring, an indicator and detection system for use with the indicator and detection composition, and a method of using the indicator and detection composition.

In an embodiment of the invention, an indicator and detection composition comprising at least one xanthene dye, and at least one component being a pH indicator is provided.

In an embodiment of the invention, an indicator and detection system comprising at least one xanthene dye, at least one component being a pH indicator, and a substrate is provided. The at least one xanthene dye is held on or in the substrate.

In an embodiment of the invention, an indicator and detection device comprising a saturated (wet-state) swab or a saturated (wet-state) substrate is provided. The swab or the substrate differentiates quaternary ammonium, hydrogen peroxide, and bleach present in a dry, surface film on a treated surface.

Further areas of applicability of the present invention will become apparent from the detailed description provided hereinafter. It should be understood that the detailed description and specific examples, while indicating the preferred embodiments of the invention, are intended for purposes of illustration only and are not intended to limit the scope of the invention.

### DESCRIPTION OF THE PREFERRED EMBODIMENTS

The following description of the embodiments of the present invention is merely exemplary in nature and is in no way intended to limit the invention, its application, or uses. The present invention has broad potential application and utility, which is contemplated to be adaptable across a wide range of industries including the healthcare and food industries. The following description is provided herein solely by way of example for purposes of providing an enabling disclosure of the invention, but does not limit the scope or substance of the invention.

The present invention provides an indicator and detection composition and system allowing the measurement of multiple disinfection/sanitization chemistries post-application to a surface and without surface damage or alteration. The indicator and detection composition and system may in one-step distinguish between multiple disinfectant chemistries. This detection is preferably indicated by a color change. The indicator and detection system solves the previously unsolved problem of being able to monitor compliance to cleaning protocols.

In an embodiment of the invention, an indicator composition is provided that comprises: at least one xanthene dye as an indicator dye, and at least one component as a pH indicator.

As to the at least one xanthene dye as an indicator dye, the indicator dye is held on or in a substrate. The dye can encounter a surface via a multitude of modalities including organic or inorganic materials. The xanthene dye is preferably a bromo-phenol blue (BPB); however, other xanthene dyes may be used.

As to the at least one component as a pH indicator, the pH indicator is stable at an acidic pH and is stable with the xanthene dye to determine the use of and distinguish between a quaternary ammonium cleaner and a sodium hypochlorite solution. Non-limiting examples of pH indicators include, but are not limited to, phenolphthalein, xylenol orange, and cresol red.

The present invention also relates to a saturated (wet state) indicator/applicator device such as a swab or a substrate. Non-limiting examples of substrates are absorbent materials including, but not limited to, foam, fabrics, and wipes. Fabrics may be, for example, woven or non-woven. The device is utilized to differentiate quaternary ammonium (Quat), H₂O₂, and bleach levels present in a dry, surface film on a treated surface. The applicator/indicator device can be pre-saturated or saturated on site before use.

The applicator/indicator device of the present invention would utilize a wet state indicator (xanthene dye with a pH indicator in the wet state) to determine the presence of a Quat, H₂O₂, or bleach on the treated surface having the dry, surface film. The applicator/indicator device can be used, for example, by hospital staff or healthcare professionals to measure compliance with cleaning protocols. This device is used to allow a hospital to understand how a room has been cleaned and which surfaces, if any, were not cleaned.

The xanthene dye is preferably a bromo-phenol blue (BPB) having a concentration in a range of 0.5 ppm to 500 ppm, more preferably in a range of 1 ppm to 50 ppm. Additionally, the pH range of a solution comprising the xanthene dye is acidic, preferably in a pH range of 0 to 4. BPB changes color and turns blue when in a complex with a quaternary ammonium compound. This compound allows the detection of quaternary ammonium.

Sodium hypochlorite is also commonly utilized in disinfection. The sodium hypochlorite is highly unstable in solution and is typically stabilized utilizing a base such as sodium hydroxide. When a surface is disinfected with sodium hypochlorite, the hypochlorite molecule readily oxidizes and leaves no trace residue on the surface. However, the stabilizing base reagent remains on the surface. Therefore, the detection of the use of sodium hypochlorite can he accomplished by measuring the residual pH of the surface.

Non-limiting examples of pH indicators include, but are not limited to, phenolphthalein, xylenol orange, and cresol red. Phenolphthalein is preferably used in a range of 50 ppm to 500 ppm. Cresol red or xylenol orange is preferably used in a range of 2 ppm to 20 ppm.

In accordance with the invention, a one step detection system utilizes a pH indicator that is stable at an acidic pH and is stable with BPB to determine the use of and distinguish between a quaternary ammonium cleaner, a hydroxide based disinfectant, and a sodium hypochlorite solution. Phenolphthalein is clear at an acidic pH and turns a pink/purple in the presence of a base. Cresol red is yellow at an acidic pH and turns red/purple in the presence of a base.

The term “or” as used in this disclosure and the appended claims is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form. Throughout the specification and claims, the following terms take at least the meanings explicitly associated herein, unless the context dictates otherwise. The meanings identified below do not necessarily limit the terms, but merely provided illustrative examples for the terms. The meaning of “a,” “an,” and “the” may include plural references, and the meaning of “in” may include “in” and “on.” The phrase “in one embodiment,” as used herein does not necessarily refer to the same embodiment, although it may.

### EXAMPLES

Experimental tests were conducted with the detection composition and system of the present invention using cotton tip applicators and separate solutions.

### Example 1

A disinfectant was placed on a glass surface and allowed to dry. Swabs were saturated with a xanthene indicator solution comprising bromo-phenol blue (BPB) that had a pH in the range of 1 to 4. The swabs were originally yellow. A color change was noted when the indicator solution interacted with both NaClO and quaternary ammonium. There was not a drastic difference in the color associated with NaClO and quaternary ammonium. The color change associated with NaClO went from yellow to blue. There was no color change associated with H₂O₂. The color change associated with quaternary ammonium went from yellow to blue. BPB was yellow at an acidic pH and turned blue.

### Example 2

The ability of the pH indicator to interact with a bleach treated surface but not a quaternary ammonium treated surface was demonstrated.

A disinfectant was placed on a glass surface and allowed to dry. Swabs were saturated with a pH indicator solution that had a pH in the range of 1 to 4. A color change was noted when the indicator solution interacted with both NaClO and quaternary ammonium. There was not a drastic difference in the color associated with NaClO and quaternary ammonium. The cresol red solution started off as a yellow color and became purple with NaClO and quaternary ammonium. The phenolphthalein solution started as clear and turned a light pink with the NaClO and quaternary ammonium samples.

### Example 3

A disinfectant was placed on a glass surface and allowed to dry. In order to differentiate between NaClO and quaternary ammonium, a combination of indicators needed to be used. Swabs were saturated with an indicator solution containing xanthene dye and a pH indicator that had a pH in the range of 1 to 4. A color change was noted when the indicator solution interacted with both NaClO and quaternary ammonium. The difference in color was apparent when the combination indicator solution, with a xanthene dye and pH indicator was utilized. The color change observed with a BPB and cresol red solution was brown-yellow to purple in the presence of NaClO, brown-yellow to yellow in the presence of H₂O₂, and brown-yellow to green in the presence of quaternary ammonium.

It will therefore be readily understood by those persons skilled in the art that the present invention is susceptible of broad utility and application. Many embodiments and adaptations of the present invention other than those herein described, as well as many variations, modifications and equivalent arrangements, will be apparent from or reasonably suggested by the present invention and the foregoing description thereof, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements.

What is claimed is:

1. An indicator and detection composition comprising:
a xanthene dye, and
a pH indicator.

2. The indicator and detection composition according to claim 1, wherein the xanthene dye is a bromo-phenol blue (BPB).

3. The indicator and detection composition according to claim 1, wherein the pH indicator is selected from the group consisting of phenolphthalein, xylenol orange, cresol red, and a combination thereof.

4. The indicator and detection composition according to claim 1, wherein the xanthene dye has a concentration in a range of 0.5 ppm to 500 ppm.

5. The indicator and detection composition according to claim 4, wherein the xanthene dye has a concentration in a range of 1 ppm to 50 ppm.

6. The indicator and detection composition according to claim 1, wherein the composition is acidic.

7. The indicator and detection composition according to claim 1, wherein the composition has a pH range of 0 to 4.

8. The indicator and detection composition according to claim 3, wherein the phenolphthalein has a concentration in a range of 50 ppm to 500 ppm.

9. The indicator and detection composition according to claim 3, wherein the cresol red or xylenol orange has a concentration in a range of 2 ppm to 20 ppm.

10. An indicator and detection system comprising:
a xanthene dye,
a pH indicator, and
a substrate,
wherein the xanthene dye is held on or in the substrate.

11. The indicator and detection composition according to claim 10, wherein the xanthene dye is a promo-phenol blue (BPB).

12. The indicator and detection composition according to claim 10, wherein the pH indicator is selected from the group consisting of phenolphthalein, xylenol orange, cresol red, and a combination thereof.

13. The indicator and detection composition, according to claim 10, wherein the xanthene dye has a concentration in a range of 0.5 ppm to 500 ppm.

14. The indicator and detection composition according to claim 13, wherein the xanthene dye has a concentration in a range of 1 ppm to 50 ppm.

15. The indicator and detection composition according to claim 10, wherein the composition is acidic.

1. The indicator and detection composition according to claim 10, wherein the composition has a pH range of 0 to 4.

17. The indicator and detection composition according to claim 12, wherein the phenolphthalein has a concentration in a range of 50 ppm to 500 ppm.

18. the indicator and detection composition according to claim 12, wherein the cresol red or xylenol orange has a concentration in a range of 2 ppm to 20 ppm.

19. An indicator and detection device comprising
a saturated swab or a saturated substrate,
wherein the saturated swab or the saturated substrate differentiates quaternary ammonium, hydrogen peroxide, and bleach present in a dry, surface film on a treated surface.

20. The indicator and detection device according to claim 19, wherein the saturated swab or the saturated substrate is saturated with a detection composition having a xanthene dye and a pH indicator.

21. The indicator and detection device according to claim 19, wherein the xanthene dye is a bromo-phenol blue (BPB).

22. The indicator and detection device according to claim 19, wherein the pH indicator is selected from the group consisting of phenolphthalein, xylenol orange, cresol red, and a combination thereof.
